(19) United States
(12) Patent Application Publication
(10) Pub. No.: US 2018/0271546 A1
(43) Pub. Date: Sep. 27, 2018

(54) Medical Catching-Wire Instrument

(71) Applicant: EPflex Feinwerktechnik GmbH, Dettingen/Erms (DE)

(72) Inventor: Bernhard UIHLEIN, Dettingen (DE)

(21) Appl. No.: 15/524,574

(22) PCT Filed: Nov. 3, 2015
(86) PCT No.: PCT/EP2015/075553

§ 371 Date: May 4, 2017

(30) Foreign Application Priority Data
Nov. 5, 2014 (DE) 10 2014 222 600.7

(51) Int. Cl. A61B 17/221

(52) U.S. Cl. CPC A61B 17/221; A61B 2090/3954; A61B 2017/2212

(57) ABSTRACT

A medical catching-wire instrument has a catching-wire basket designed to be axially movable relative to an instrument tube between an unfolded state, in which the basket is moved distally forward out of the instrument tube, and a folded state, in which the basket is moved proximally rearward into the instrument tube. A control tube can be received in the instrument tube and is axially movable relative to the latter. The control tube is coupled in a distal end area to a proximal end area of the basket. The catching-wire basket has a distal enclosing portion which is more close-meshed than a proximally adjoining portion, and/or the basket has a proximal expanding portion which is more close-meshed than a distally adjoining portion, and/or the basket is constructed from wire segments which are substantially rectangular in cross section and which, when the basket is unfolded, extend at least in a basket subregion with a non-tangential broad side of the wire.

### BACKGROUND AND SUMMARY OF THE INVENTION

The invention relates to a medical catching-wire instrument with a catching-wire basket which is designed to be axially moveable relative to an instrument tube between an unfolded state, in which said catching-wire basket is moved distally forward out of the instrument tube, and a folded state, in which said catching-wire basket is moved proximally rearward into the instrument tube, and with a control tube which can be received in the instrument tube and is axially movable relative to the latter, which control tube is coupled in a distal end area to a proximal end area of the catching-wire basket. The invention relates more specifically to a catching-wire instrument of the so-called over-the-wire type, in which the catching-wire basket and the control tube are designed to be able to be pushed axially movably onto a guide wire.

A catching-wire instrument of this kind is disclosed in laid-open specification WO 93/19679 A1 and is sold by the company Teleflex Medical as a percutaneous thrombolysis instrument for removal of clots. In this known instrument, the catching-wire basket and the control tube are designed to be set in rotation, during operation, by an external drive of an associated catheter system in order to fragment thrombotic material in an affected tissue channel. For this purpose, the control tube forms a flexible drive shaft, of which the proximal end is coupled to the external drive. The catching-wire basket is formed from a plurality of individual wire pieces which extend all the way from a proximal end to a distal end of the catching-wire basket and are gathered at each end, and they can optionally have a twisted profile about a longitudinal axis of the basket. It is also optional for the basket-forming wire pieces to be provided with a cutting edge in order to increase the fragmentation effect of the rotating wire basket.

Patent publication DE 10 2004 055 375 B4 discloses a catching-wire basket which is cut in one piece from a tube piece.

Laid-open publication WO 2014/086917 A1 discloses a medical catching-wire instrument with a special material combination of a catching-wire basket, an instrument tube receiving the latter, and a rod-shaped control element guided in the latter. The catching-wire basket can be cut in one piece from a tube piece. Furthermore, the instrument can be provided with what is called an MR marker in order to make it visible in magnetic resonance (MR) applications, especially in nuclear magnetic resonance (NMR) applications or magnetic resonance tomography (MRT) applications. The MR marker can be located on the catching-wire basket and/or on the control element and/or on the instrument tube, in each case along the entire length of the instrument component in question or only along part thereof.

An object addressed by the invention is that of providing a medical catching-wire instrument of the type mentioned at the outset which is further improved over the prior art explained above and in particular can be realized with good capture properties and/or with relatively low manufacturing effort.

The invention achieves this object by providing a medical catching-wire instrument comprising a catching-wire basket which is designed to be axially moveable relative to an instrument tube between an unfolded state, in which said catching-wire basket is moved distally forward out of the instrument tube, and a folded state, in which said catching-wire basket is moved proximally rearward into the instrument tube, and a control tube receivable in the instrument tube and axially movable relative to the latter, which control tube is coupled in a distal end area to a proximal end area of the catching-wire basket, wherein the catching-wire basket and the control tube are designed to be pushed axially movably onto a guide wire.

According to one aspect of the invention, the catching-wire basket has a distal enclosing portion which is more close-meshed than a proximally adjoining portion. With this measure, the wire basket is divided into an enclosing portion which, by virtue of its close meshes, allows captured particles to be safely enclosed and secured, and a proximally adjoining portion which, by virtue of its greater mesh width, facilitates the capture of particles. According to a further aspect of the invention, the catching-wire basket has a proximal expanding portion which is more close-meshed than a distally adjoining portion. Such an expanding portion can support the unfolding behavior of the catching-wire basket and contribute to an advantageous shaping of the wire basket in the unfolded state. According to yet another aspect of the invention, the catching-wire basket is constructed from wire segments which are substantially rectangular in cross section and which, when the catching-wire basket is unfolded, extend at least in a basket subregion with non-tangential broad side of the wire. This means that the wire segments in this area point radially outward with a narrow side, or with one of their edges, and not with their broad side, as a result of which, for example, it can be made easier to remove material such as clot material that adheres to an inner wall of a body tissue channel.

In a development of the invention, the distal enclosing portion is more close-meshed than the proximal expanding portion. Captured particles can be readily secured in the more close-meshed enclosing portion, and the expanding portion can be used to obtain a corresponding desired shape of the wire basket in the unfolded state.

In a development of the invention, the catching-wire basket has a capture portion between the enclosing portion and the expanding portion, which capture portion is more wide-meshed than the enclosing portion and the expanding portion. By virtue of this basket design, it is possible to further optimize the properties of the catching-wire basket in terms of its ability to safely capture and secure particles, for example particles of thrombotic material that are to be dislodged and removed.

In a development of the invention, the control tube has an internal diameter corresponding substantially to an external diameter of the guide wire, in such a way that, in its position when pushed onto the guide wire, it bears against the guide wire about the entire circumference and is axially movable with respect to the guide wire. The control tube can thus be guided on the guide wire over practically the entire surface when it is pushed onto the guide wire and moved along same.

In a development of the invention, the catching-wire basket is cut in one piece from a tube piece. This permits production with relatively low costs, and the wire basket can be realized with very advantageous configurations for achieving an optimal function as regards the capturing and securing of particles.

In a development of the invention, the catching-wire basket terminates proximally with a rear end sleeve, which is fixed with an overlap on a distal end area of the control tube. This constitutes a very functionally safe and reliable coupling of the catching-wire basket to the control tube. If the catching-wire basket is cut from a tube piece, the end sleeve can be formed by a tubular stub of the tube piece, which keeps the production effort low.

In a development of the invention, the catching-wire basket terminates distally with a front end sleeve, which is fixed with an overlap on a proximal end area of a guide tube sleeve. Once again, the front end sleeve can be formed from a tubular stub of the tube piece when the catching-wire basket is cut from a tube piece. The guide tube sleeve can assist the guiding of the catching-wire basket in the axial movement thereof relative to the guide wire, when the catching-wire basket is pushed onto the guide wire for use.

In a development of the invention, the instrument tube and/or the control tube and/or the catching-wire basket and/or the guide tube sleeve is provided at least partially with an MR marker. The MR visibility of the instrument can thus be improved specifically in a desired region.

In a development of the invention, the catching-wire instrument comprises a guide wire onto which the catching-wire basket and the control tube can be pushed axially movably and which thus completes the instrument.

### BRIEF DESCRIPTION OF THE DRAWINGS

Advantageous embodiments of the invention are shown in the drawings and are described below. In the drawings:

FIG. 1 shows a schematic side view of a catching-wire basket for a medical catching-wire instrument of the over-the-wire type,

FIG. 2 shows a view, corresponding to FIG. 1, of a catching-wire basket with a modified basket shape,

FIG. 3 shows a schematic developed view of a tube cut pattern for producing the catching-wire basket of FIG. 1,

FIG. 4 shows a view, corresponding to FIG. 3, for the production of the catching-wire basket of FIG. 2,

FIG. 5 shows a detailed cross-sectional view along a line V-V in FIG. 1,

FIG. 6 shows a schematic sectional view of a thrombotic tissue channel,

FIG. 7 shows the view from FIG. 6 with a guide wire pushed through the thrombotic area,

FIG. 8 shows the view from FIG. 7, with the catching-wire basket of FIG. 1 pushed onto the guide wire and deployed in a position behind the thrombotic area,

FIG. 9 shows a schematic longitudinal sectional view of a connection area of the catching-wire basket and of a control tube pushed proximally onto an end sleeve thereof,

FIG. 10 shows a transverse sectional view along a line X-X in FIG. 9,

FIG. 11 shows the view of FIG. 9 for a variant with a control tube pushed proximally into an end sleeve of the catching-wire basket,

FIG. 12 shows a transverse sectional view along a line XII-XII in FIG. 11,

FIG. 13 shows a detailed longitudinal sectional view of a distal end area of a catching-wire basket,

FIG. 14 shows the view of FIG. 13 for a variant with a guide tube sleeve pushed on distally,

FIG. 15 shows the view of FIG. 13 for a variant with a guide tube sleeve pushed in distally,

FIG. 16 shows a schematic longitudinal sectional view of a distal part of an MR-visible guide wire with a catching-wire basket/control tube unit pushed on,

FIG. 17 shows a schematic longitudinal sectional view of a distal part of a catching-wire basket/control tube unit, with MR markers arranged proximally and distally of the basket,

FIG. 18 shows the view of FIG. 17 for a variant with MR markers mounted on the wire pieces of the wire basket, and

FIGS. 19 to 24 show schematic side views of further usable catching-wire baskets of different configurations.

### DETAILED DESCRIPTION OF THE DRAWINGS

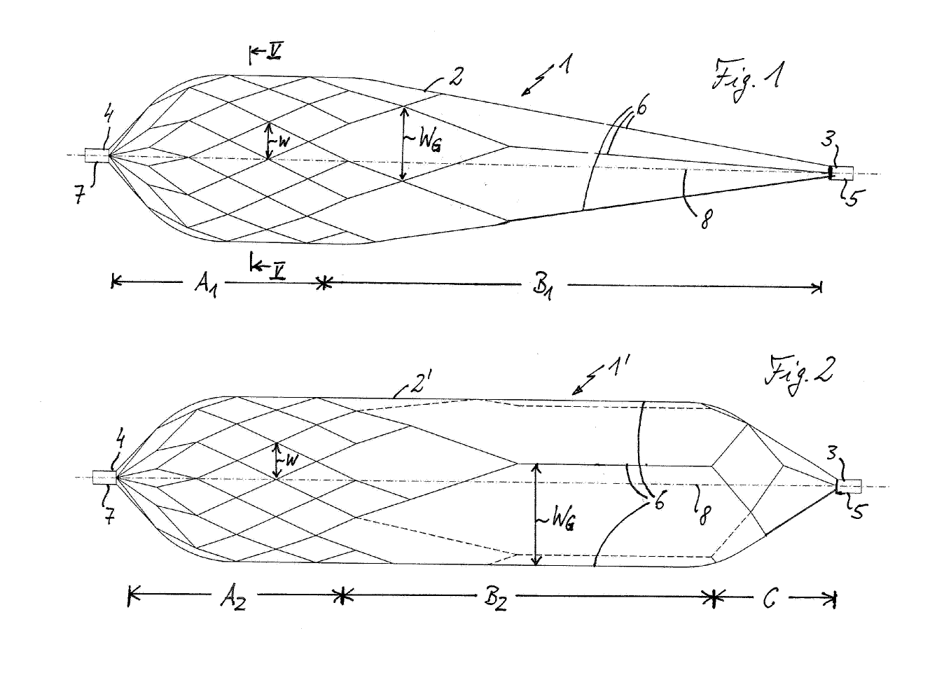

A catching-wire basket 1 shown schematically in the unfolded state in FIG. 1 is constructed from a wire structure 2 that extends between a proximal end area 3 and a distal end area 4. The wire basket 1 is divided into a distal enclosing portion A₁ which is relatively close-meshed with a maximum opening or mesh width w, and a proximally adjoining portion B₁ with, in comparison, a greater maximum opening or mesh width W_{G}. The proximal end area 3 of the wire basket 1 is formed by a rear end sleeve 5 from which, in order to form a basket, a plurality of wire segments 6 extend with an axial and a radial component in the unfolded state illustrated. In distal direction, the wire segments 6 branch repeatedly, as a result of which the braid-like wire structure 2 shown is formed.

The structure 2 is chosen such that the wire basket in the distal enclosing portion A₁ is substantially more close-meshed than in the proximal portion B₁, i.e. has a much smaller mesh width w, for example a mesh width w that is at most half as great or at most a third as great as the mesh width W_{G} of the proximal portion B₁. At the distal end area, the corresponding wire segments run together again, in the example shown specifically to a front end sleeve 7 forming the distal basket termination. A guide wire can be pushed through the rear end sleeve 5 and through the front end sleeve 7 along a basket longitudinal axis 8, making the wire basket 1 suitable for use in a catching-wire instrument of the over-the-wire type. Particles to be captured, e.g. thrombotic particle material, can be easily captured by the unfolded basket 2 in the proximal portion B₁ by virtue of the relatively large mesh width thereof. Thereafter, the particles can be safely secured in the more close-meshed distal enclosing portion A₁ with its smaller mesh width w and can be enclosed such that they do not emerge again out of the basket 2.

The catching-wire basket 1 of FIG. 1 can advantageously be produced in one piece from a tube piece, in particular by being cut from the tube piece, e.g. by laser cutting. For this purpose, the tube piece is cut in a corresponding cut pattern or slit pattern. FIG. 3 shows an accordingly cut or slit tube piece 9 in a developed view. It can be clearly seen from this how the wire structure cut from the tube piece 9 comprises a plurality of wire segments 6 which issue from the proximal end area 3 and then branch repeatedly in order to form the proximal capture portion B₁, which then merges into the distal enclosing portion A₁, which is formed by a comparatively finer slit pattern. A respective end stub of the tube piece forms the proximal, rear end sleeve 3 or the distal, front end sleeve 4 of the wire basket.

FIGS. 2 and 4, in views corresponding to FIGS. 1 and 3 respectively, illustrate a further catching-wire basket that can be used according to the invention, with a basket design that is modified compared to that of FIGS. 1 and 3. To make matters clearer here, and in the subsequent figures, the same reference signs are used for identical or functionally equivalent elements, and so the description of these elements can accordingly be obtained by reference to the explanations concerning the figures that have already been described beforehand.

By comparison with the structure of the basket 1 of FIG. 1, a catching-wire basket 1′ shown in FIG. 2 has a braid-like wire structure 2′ that is modified such that it has a substantially constant external diameter along most of its length. Specifically, it is divided into a proximal expanding portion C, a distal enclosing portion A₂ and, between these, a substantially cylindrical capture portion B₂. The enclosing portion A₂, with a comparatively small mesh width w, corresponds substantially to that of the basket 1 of FIG. 1. The capture portion B₂ is again the basket portion with the maximum mesh width W_{G} for easily passing and thereby catching particles into the basket 1′ transversely with respect to the basket longitudinal axis 8. In the basket 1′ of FIG. 2, this capture portion B₂ is adjoined by the proximal expanding portion C, in which the wire segments issuing from the proximal end area 3 already branch after a relatively short length, such that the basket 1′, already at a relatively short axial distance from the proximal end area 3, widens to its maximum basket diameter and merges into the capture portion B₂. The expanding portion C thus supports the unfolding of the basket 1′, such that the basket 1′ already attains its maximum diameter after a short axial length. Moreover, the branched expanding portion C stabilizes this basket configuration toward the proximal end area 3.

In this way, the basket 1′ of FIG. 2 acquires a somewhat cylindrical shape, whereas the basket 1 of FIG. 1 has a conically widening wire structure over a majority of its length, and in particular over most of its capture portion B₁. The catching-wire basket 1′ of FIG. 2 can also be cut in one piece from a tube piece. This is shown in FIG. 4 in an associated developed view of a corresponding tube piece 9′. From a comparison of FIGS. 3 and 4, in particular the modified basket shape in the expanding portion C of the basket 1′ of FIG. 2 can be seen. Otherwise, the same properties and advantages indicated above for the catching-wire basket 1 of FIG. 1 apply also to the catching-wire basket 1′ of FIG. 2.

To form the finished wire basket 1, 1′, the associated, ready-cut tube piece 9, 9′ is compressed lengthwise, as a result of which it unfolds to give the desired wire basket 1, 1′. For the tube piece 9, 9′, a superelastic material is preferably used, for example as a nickel-titanium alloy, such that the finished catching-wire basket 1, 1′ can be brought elastically from the unfolded state to a folded state and can be returned from the latter to the unfolded state again. For this purpose, it is moved during use into a surrounding instrument tube and thus folded up, and it can then be moved forward out of the latter again and thus unfolded. This is known per se to a person skilled in the art and needs no further explanation here.

It will be appreciated that other desired basket configurations can also be produced in one piece by cutting from a tube piece, for which purpose the cutting pattern is suitably selected in each case, as is known per se from the pertinent prior art mentioned above.

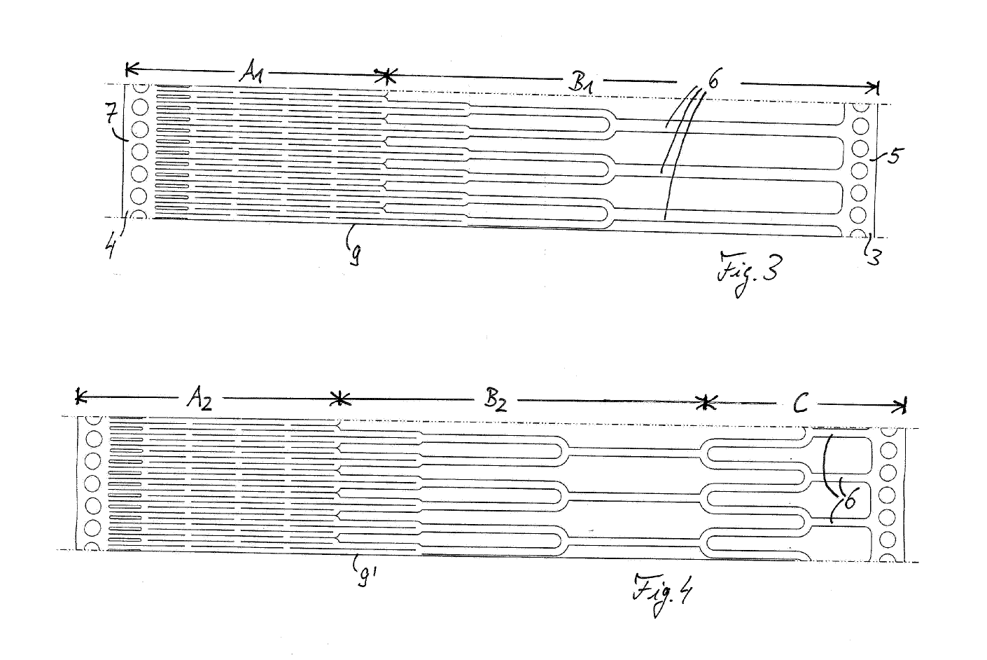

If the catching-wire basket is produced by suitable cutting of a tube piece, the individual wire pieces or wire segments 6 can have a substantially rectangular cross section, wherein their cross-sectional width corresponds to the tube wall thickness of the tube piece, while their cross-sectional length corresponds to the slit distance between two adjacent slits cutting the wire segment or wire piece from the tube piece. Depending on the desired dimensions, the cross-sectional width can be smaller or greater than the cross-sectional length. FIG. 5 depicts an illustrative embodiment with a cross-sectional width b of the wire segments 6, which is smaller than their cross-sectional length 1.

By suitable dimensioning of the present wire basket structures, it is possible that the wire segments or wire pieces 6 twist to a certain extent about their longitudinal axis when the basket is brought to its unfolded state. In the example of FIG. 5, this is represented by a twist angle β about which the wire segments 6 in the basket area in question are each twisted from their starting position, which is present after slitting and cutting the tube piece 9, 9′ and before unfolding the wire basket, to their position shown in FIG. 5 with the wire basket 1, 1′ unfolded.

In the starting position, the wire segments 6 of rectangular cross section lie with their main sides H parallel to the associated tangential plane of the tube piece 9, 9′, whereas the narrow sides S formed by the cutting edges lie in radial planes perpendicular thereto. The tangential planes to the tube piece 9, 9′ correspond to parallel tangential planes of the basket circumference KU indicated by dot-and-dash lines in the detail of FIG. 5. Consequently, when the wire basket 1, 1′ is unfolded, the wire segments 6 in this case lie in corresponding basket areas with their main sides H not parallel to the basket circumference KU, and instead they are twisted by the angle β about their longitudinal axis. This moreover has the effect that the wire segments 6, as can be seen from FIG. 5, point radially outward with one of their two narrow sides S or with an edge line KL adjoining the latter. These outwardly facing wire edges KL or outwardly facing narrow sides S of the wire segments 6, 6′ can advantageously strengthen a dislodging action if the wire basket 1, 1′ is used with its outside to dislodge material which adheres to a body tissue channel and which, after being dislodged, is intended to be caught in the catching basket and held securely therein. This strengthening action, compared to a wire basket in which the wire segments do not twist in this way upon unfolding of the basket, or in which wire segments of circular cross section are used, is thus obtained without additional wire-processing measures, e.g. additional grinding of the wire segments in order to obtain a dislodging edge of greater or lesser sharpness.

FIGS. 6 to 8 illustrate a typical case of use of the catching-wire instrument according to the invention. In the example shown, the catching-wire basket 1 of FIG. 1 is used which is fixed at the distal end of a control tube 14. FIG. 6 shows an area of a body tissue channel 10 which is to be treated and on the inner wall of which a clot has formed typically by deposition of thrombotic material 11, which clot reduces the effective cross section of the tissue channel 10. Firstly, a guide wire 12 is pushed into the tissue channel 10 and through the clot 11 and advanced beyond the latter, as is shown in FIG. 7.

The unit composed of catching-wire basket 1 and control tube 14 with surrounding instrument tube 13 is then pushed onto the guide wire 12 from the direction of the proximal end thereof and is advanced along it until the instrument tube 13 is located with a distal portion, in which the catching-wire basket 1 is received in its folded state, behind the clot 11. The catching-wire basket 1 is then moved distally forward out of the instrument tube 13, as a result of which it widens into its unfolded state as shown in FIG. 8. For this purpose, while the instrument tube 13 is held securely, the control tube 14 along with the distally coupled catching-wire basket 1 can be pushed axially forward, or, while the control tube 14 and catching-wire basket 1 are held securely, the instrument tube 13 is pulled back until it releases the basket 1. Operating elements which can be used for these control movements, and to which the instrument tube 13 and the control tube 14 are coupled with their proximal end area, are known per se to a person skilled in the art and therefore need no further explanation here.

Proceeding from the position of the deployed catching-wire basket 1 behind the clot 11 as shown in FIG. 8, the catching-wire basket/control tube unit 1, 14, together with instrument tube 13, is moved back axially along the guide wire 12. As soon as the wire basket 1 reaches the clot 11, it can detach or dislodge the latter from the inner wall of the tissue channel 10, and the dislodged thrombotic material 11 can pass through the wide meshes of the capture portion B₁ into the wire basket 1, where it is safely retained by the more close-meshed enclosing portion A₁ and remains captured. If necessary, the wire basket 1 can then be moved again into the instrument tube 13 to the extent that the particles captured in it are tightly enclosed by its wire braid. This is effected by the control movement that is the inverse of the above-described movement of the wire basket 1 out of the instrument tube 13, i.e. by active pulling back of control tube 14 and wire basket 1 or active advancing of the instrument tube 13. Finally, the control tube/wire basket unit 1, 14, together with the surrounding instrument tube 13 with the particles captured in the wire basket 1, is moved out of the tissue channel 10. Depending on the particular use, the guide wire 12 is also withdrawn again out of the tissue channel 10, or it can alternatively remain therein.

The described dislodging and capturing of the thrombotic material 11 is assisted by the particular, subdivided configuration of the catching-wire basket 1 with its capture portion B₁ and its comparatively more close-meshed enclosing portion A₁. Effective release of material that may be adhering more strongly to the inner wall of the tissue channel 10 can be assisted by the twisted position of the wire segments 6 of rectangular cross section as described above with reference to FIG. 5, and as may be present at least in a subregion of the basket in corresponding embodiments. For example, this can be a subregion in which the wire basket 1 has its maximum basket diameter, as in the transition area between enclosing portion A₁ and capture portion B₁ in the basket 1 of FIG. 1, or, as in the basket 1′ of FIG. 2, additionally over practically the entire capture portion B₂ proximally up to the expanding portion C. The narrow sides S or wire segment edges KL turned radially outward in this way provide a non-smooth profile or edged profile of the outer circumference of the wire basket 1, which profile can have a material-dislodging effect. Therefore, in the present catching-wire instrument, the catching-wire basket 1, 1′ for dislodging material adhering to a tissue channel, for example the thrombotic material 11 of FIGS. 6 to 8, does not have to be set in a rotation movement adapted for this purpose. It suffices to simply pull back the unfolded basket 1, 1′, which if necessary can be assisted by turning it gently by hand back and forth about an angle of less than 360°.

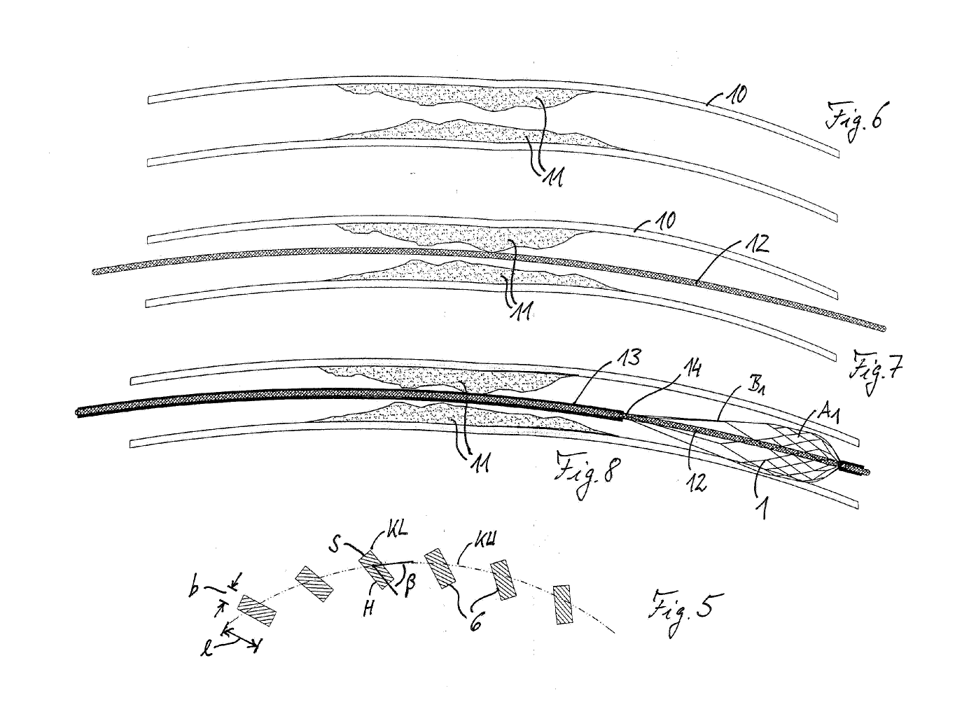

FIGS. 9 to 15 show advantageous possible embodiments of the control tube/wire basket coupling and of the front, distal wire basket configuration, taking the example of a catching-wire basket 1₁ which is cut in one piece from a tube piece and which can for example be of one of the two basket types that have been explained above with reference to FIGS. 1 and 2. The wire basket 1₁ is preferably cut in one piece from a tube section, and its wire segments 6 terminate proximally in a rear end sleeve 5₁ and distally in a front end sleeve 7₁, as described above in connection with the baskets 1, 1′ of FIGS. 1 and 2.

In the illustrative embodiment of FIGS. 9 and 10, the catching-wire basket is fixed proximally by its rear end sleeve 5₁ with an overlap on a distal end area of a control tube 14₁ such that the control tube 14₁ with its distal end area encloses the rear end sleeve 5₁ to be in tight contact therewith. For this purpose, the control tube 14₁ can be shrunk for example onto the end sleeve 5₁; alternatively, it is fixedly connected thereto in another way in the overlap area, e.g. by adhesive bonding. The rear end sleeve 5₁ leaves a through-channel 15₁ for the passage of a guide wire.

In the illustrative embodiment of FIGS. 11 and 12, the rear end sleeve 5₁ of the wire basket 1₁ is pushed onto the distal end area of a control tube 14₂. Once again, the proximal end sleeve 5₁ of the wire basket 1₁ and the distal end area of the control tube 14₂ are fixed firmly to each other, e.g. by adhesive bonding. The control tube 14₂ in this case determines the diameter of a corresponding through-channel 15₂ for a guide wire that is to be guided through.

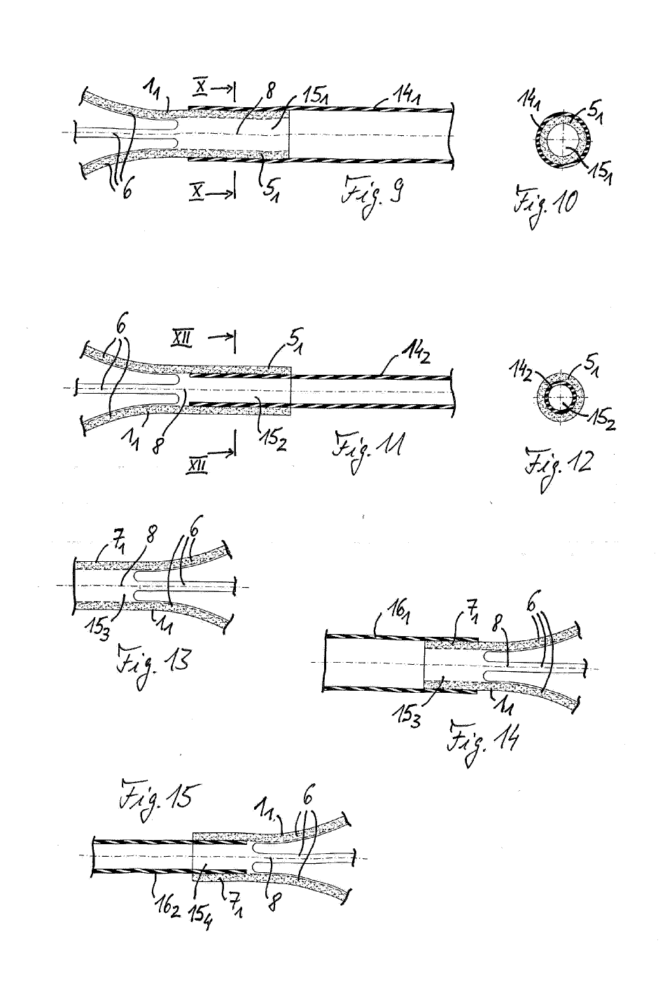

FIG. 13 shows the front, distal end area of the wire basket 1₁, in which the basket-forming wire segments 6 run together in a front, distal end sleeve 7₁. The distal end sleeve 7₁ leaves a through-channel 15₃ for the passage of a guide wire along the basket longitudinal axis 8.

In the illustrative embodiment of FIG. 14, a distal guide tube sleeve 16₁ is provided which is fixed with a proximal end area to the front end sleeve 7₁ in overlapping manner. For this purpose, it surrounds the front end sleeve 7₁ in the example shown and tightly encloses the latter, e.g. by shrinkage. Alternatively, the guide tube sleeve 16₁ is firmly connected in another way in the overlap area to the distal end sleeve 7₁, e.g. by adhesive bonding.

In the illustrative embodiment of FIG. 15, a guide tube sleeve 16₂ is provided, onto the proximal end area of which the distal end sleeve 7₁ of the wire basket 1₁ is fitted. The distal end sleeve 7₁ and the guide tube sleeve 16₂ are fixed firmly to each other in the overlap area, e.g. by adhesive bonding. In this illustrative embodiment, the guide tube sleeve 16₂ defines a through-channel 15₄ for the passage of a guide wire in this area.

In the illustrative embodiments of FIGS. 14 and 15, the guide tube sleeve 16₁, 16₂ can assist in guiding the catching-wire basket 1₁ on a guide wire onto which the wire basket 1₁ is pushed during use. The guide tube sleeve 16₁, 16₂ can be produced from the same material or tube material as the control tube 14₁, 14₂. The latter then automatically provides a through-channel of the same diameter for a guide wire to be guided through both in the proximal instrument area, in which the control tube 14₁, 14₂ is located, and in the distal area of the wire basket 1₁. Alternatively, however, it is possible to use different tubes or materials for the control tube on the one hand and for the guide tube sleeve on the other hand.

FIG. 16 shows an illustrative embodiment with the catching-wire basket 1₁ in a position pushed onto a guide wire 12₁ in the embodiment variant with the control tube 14₂ according to FIGS. 11 and 12 and with the distal guide tube sleeve 16₂ according to FIG. 15. The guide wire 12₁ has a known structure composed of a core wire 17 which, except for a distal end area, is tightly surrounded by a more flexurally stiff envelope 18. In the distal end area, this envelope 18 is adjoined by a flexurally softer envelope 19, as is likewise known per se. The guide wire 12₁ is designed for use in MR applications, for which purpose the core wire 17 is preferably made of a plastic material and, in the distal end area, an MR marker 20 is provided which in this case is formed by an MR overlay on the core wire 17, which MR overlay is embedded in the flexurally softer envelope 19.

As can be seen from FIG. 16, the internal diameter of the control tube 14₂ and of the distal guide tube sleeve 16₂ corresponds substantially to the external diameter of the guide wire 12₁ in such a way that the control tube 14₂ and the guide tube sleeve 16₂ bear against the guide wire 12₁ about the entire circumference but remain axially movable relative thereto with a sufficiently low actuation force. Alternatively, for suitable application cases, provision can be made to leave a small annular gap between control tube 14₂ or guide tube sleeve 16₂ on the one hand and guide wire 12₁ on the other hand. As can also be seen from FIG. 16, the guide tube sleeve 16₂ improves the guiding of the catching-wire basket/control tube unit during the movement thereof along the guide wire 12₁. For the sake of clarity, the instrument tube in which the wire basket/control tube unit is received has been omitted in FIG. 16.

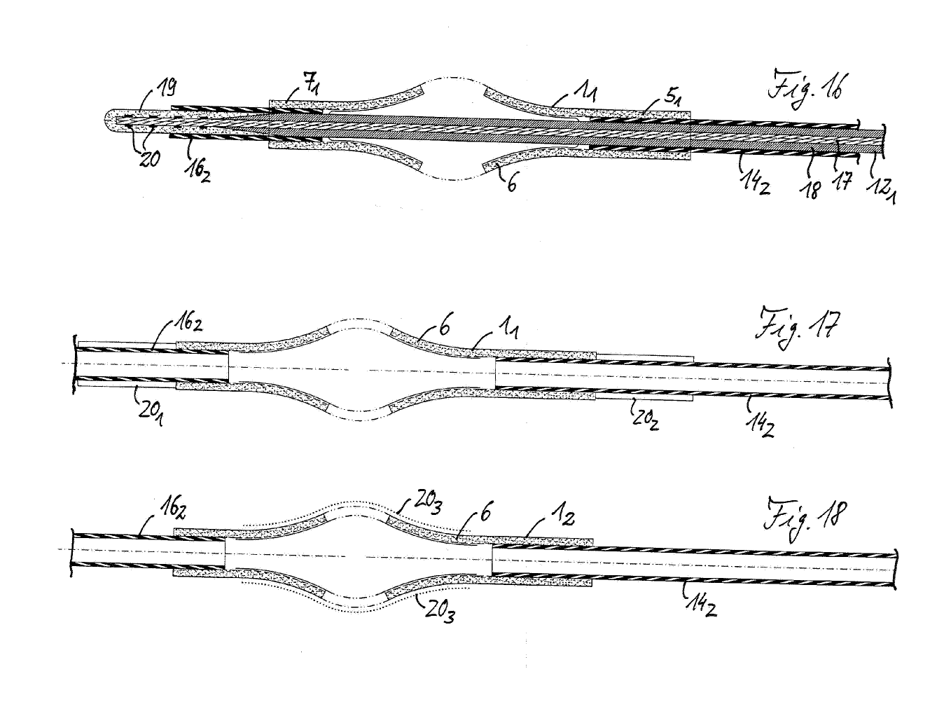

It will be appreciated that, if necessary, the catching-wire basket/control tube unit and/or the instrument tube can also be provided with an MR marker. By way of example, FIGS. 17 and 18 show two corresponding illustrative embodiments as variants of the catching-wire basket/control tube unit of FIG. 16. In the illustrative embodiment of FIG. 17, an MR marker 20₁ is applied to the outside of the guide tube sleeve 16₂ in the area in front of the distal end of the catching-wire basket 1₁. In addition, an MR marker 20₂ is applied to the control tube 14₂ in a portion that proximally adjoins the proximal end of the catching-wire basket 1₁.

In the illustrative embodiment of FIG. 18, an MR marker 20₃ is provided on a catching-wire basket 1₂ which has been modified in terms of the fact that its basket-forming wire segments 6 are coated with the MR marker material, as is represented by dots in FIG. 18. It will be appreciated that any other overlays/coatings of catching-wire basket, control tube and/or optional guide tube sleeve with MR marker material can be provided in order, if necessary, to improve the visibility of the instrument in MR applications, at one or more points, in one or more areas or over the entire length of the instrument. For this purpose, the instrument tube can also be provided in a manner known per se with a suitable MR marker coating, in which connection reference may be made for example to corresponding designs of an instrument tube or of an instrument tube sleeve of the kind disclosed in WO 2014/086917 A1 mentioned at the outset. In each of the cases mentioned above, the MR marker can be configured as a so-called active MR marker or alternatively as a so-called passive MR marker, as is customary to a person skilled in the art.

It will also be appreciated that, in addition to the basket configurations as shown in FIGS. 1 and 2, the invention covers numerous other basket configurations with different basket shapes, depending on the requirements and the application case. In this connection, FIGS. 19 to 24 illustrate examples of some other possible embodiment variants which can all be produced in one piece, if so desired, by cutting from a tube piece. If necessary, it is also possible for the basket variants according to FIGS. 19 to 24 to be provided partially or completely with an MR marker in order to improve their MR visibility.

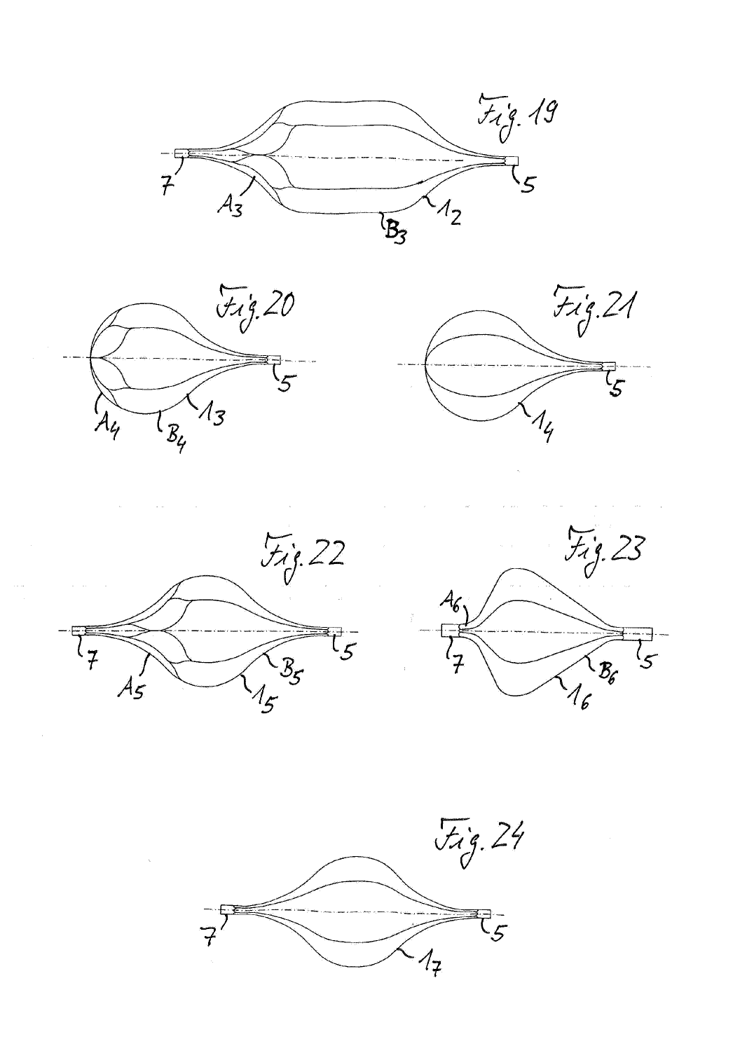

FIG. 19 shows a catching-wire basket 1₂ in which a capture portion B₃ opens out in a cup shape starting from the proximal end sleeve 5 and then extends cylindrically and merges into a rosette-shaped enclosing portion A₃ before the distal end sleeve 7. FIG. 20 shows a catching-wire basket 1₃ with a balloon-like capture portion B₄ which merges into a rosette-shaped enclosing portion A₄ with a so-called tipless end. Tipless configurations of this kind are known per se and are preferred for certain uses. FIG. 21 shows a uniformly balloon-shaped catching-wire basket 1₄, likewise with a tipless distal end, wherein this wire basket 1₄, like most of the other present baskets, is constructed from wire segments of substantially rectangular cross section which, in the unfolded state of the wire basket as shown, extend at least in a subregion of the wire basket 1₄ with a non-tangential wire broad side, as has been explained above with reference to FIG. 5.

FIG. 22 shows a catching-wire basket 1₅ with a balloon-shaped or bell-shaped capture portion B₅ which merges into a rosette-shaped enclosing portion A₅ similar to that of the basket 1₂ of FIG. 19. FIG. 23 shows a catching-wire basket 1₆ with a capture portion B₆ which widens in a pear shape and which merges into a relatively short enclosing portion A₆. FIG. 24 shows a catching-wire basket 1₇ with a quasi center-symmetrical onion shape, wherein this wire basket 1₇ is again constructed from wire segments which are of substantially rectangular cross section and which, in the unfolded state of the basket as shown, extend at least in a basket subregion with a non-tangential wire broad side, as has been explained above with reference to FIG. 5.

1-9. (canceled)

10. A medical catching-wire instrument, comprising:
a catching-wire basket which is designed to be axially moveable relative to an instrument tube between an unfolded state, in which said catching-wire basket is moved distally forward out of the instrument tube, and a folded state, in which said catching-wire basket is moved proximally rearward into the instrument tube, and
a control tube receivable in the instrument tube and axially movable relative to the latter, which control tube is coupled in a distal end area to a proximal end area of the catching-wire basket,
wherein the catching-wire basket and the control tube are designed to be pushed axially movably onto a guide wire, and
wherein the catching-wire basket comprises at least one of: a distal enclosing portion which is more close-meshed than a proximally adjoining portion, and a proximal expanding portion which is more close-meshed than a distally adjoining portion.

11. The medical catching-wire instrument as claimed in claim 10, wherein the catching-wire basket is constructed from wire segments which are substantially rectangular in cross section and which, when the catching-wire basket is unfolded, extend at least in a basket subregion with non-tangential broad side of the wire.

12. The medical catching-wire instrument as claimed in claim 10, wherein the distal enclosing portion is more close-meshed than the proximal expanding portion.

13. The medical catching-wire instrument as claimed in claim 10, wherein the catching-wire basket comprises a capture portion between the enclosing portion and the expanding portion, wherein the capture portion is more wide-meshed than the enclosing portion and the expanding portion.

14. The medical catching-wire instrument as claimed in claim 10, wherein the control tube comprises an internal diameter corresponding substantially to an external diameter of the guide wire, in such a way that, in its position when pushed onto the guide wire, it bears against the guide wire about the entire circumference and is axially movable with respect to the guide wire.

15. The medical catching-wire instrument as claimed in claim 10, wherein the catching-wire basket is cut in one piece from a tube piece.

16. The medical catching-wire instrument as claimed in claim 10, wherein the catching-wire basket terminates proximally with a rear end sleeve, which is fixed with an overlap on a distal end area of the control tube.

17. The medical catching-wire instrument as claimed in claim 10, wherein the catching-wire basket terminates distally with a front end sleeve, which is fixed with an overlap on a proximal end area of a guide tube sleeve.

18. The medical catching-wire instrument as claimed in claim 10, wherein at least one of: the instrument tube, the control tube, the catching-wire basket, and the guide tube sleeve, is provided at least partially with an MR marker.

19. The medical catching-wire instrument as claimed in claim 10, further comprising a guide wire onto which the catching-wire basket and the control tube can be pushed axially movably.

20. A medical catching-wire instrument, comprising:
a catching-wire basket which is designed to be axially moveable relative to an instrument tube between an unfolded state, in which said catching-wire basket is moved distally forward out of the instrument tube, and a folded state, in which said catching-wire basket is moved proximally rearward into the instrument tube, and
a control tube receivable in the instrument tube and axially movable relative to the latter, which control tube is coupled in a distal end area to a proximal end area of the catching-wire basket,
wherein the catching-wire basket and the control tube are designed to be pushed axially movably onto a guide wire, and
wherein the catching-wire basket is constructed from wire segments which are substantially rectangular in cross section and which, when the catching-wire basket is unfolded, extend at least in a basket subregion with non-tangential broad side of the wire.

21. The medical catching-wire instrument as claimed in claim 20, wherein the control tube comprises an internal diameter corresponding substantially to an external diameter of the guide wire, in such a way that, in its position when pushed onto the guide wire, it bears against the guide wire about the entire circumference and is axially movable with respect to the guide wire.

22. The medical catching-wire instrument as claimed in claim 20, wherein the catching-wire basket is cut in one piece from a tube piece.

23. The medical catching-wire instrument as claimed in claim 20, wherein the catching-wire basket terminates proximally with a rear end sleeve, which is fixed with an overlap on a distal end area of the control tube.

24. The medical catching-wire instrument as claimed in claim 20, wherein the catching-wire basket terminates distally with a front end sleeve, which is fixed with an overlap on a proximal end area of a guide tube sleeve.

25. The medical catching-wire instrument as claimed in claim 20, wherein at least one of: the instrument tube, the control tube, the catching-wire basket, and the guide tube sleeve, is provided at least partially with an MR marker.

26. The medical catching-wire instrument as claimed in claim 20, further comprising a guide wire onto which the catching-wire basket and the control tube can be pushed axially movably.
